(19) United States
(12) Patent Application Publication
(10) Pub. No.: US 2022/0290250 A1
(43) Pub. Date: Sep. 15, 2022

(54) Genomic-driven targeted therapies

(71) Applicant: Nantomics LLC, Culver City, CA (US)

(72) Inventor: Christopher W. Szeto, Culver City, CA (US)

(73) Assignee: Nantomics LLC, Culver City, CA (US)

(21) Appl. No.: 17/635,616

(22) PCT Filed: Jul. 23, 2020
(86) PCT No.: PCT/US20/43275

§ 371 Date: Feb. 15, 2022

Related U.S. Application Data
(60) Provisional application No. 62/951,886, filed on Dec. 20, 2019.
(60) Provisional application No. 62/890,464, filed on Aug. 22, 2019.

(51) Int. Cl. C12Q 1/6886; A61P 35/00; G16B 20/20

(52) U.S. Cl. CPC C12Q 1/6886; A61P 35/00; G16B 20/20; C12Q 2600/158; C12Q 2600/156; C12Q 2600/106

(57) ABSTRACT

The present disclosure provides methods and systems of identifying a tumor patient for treatment with a combination of targeted therapy and immune oncology based on differential checkpoint expression patterns, and their association with mutation status, irrespective of the tumor tissue type. Also provided herein are methods of treatment for a tumor with a combination of targeted therapy and immune-oncology (IO) therapy.

This application claims priority to our co-pending US provisional patent applications with the Ser. No. 62/890,464 which was filed Aug. 22, 2019, and 62/951,886 which was filed Dec. 20, 2019. Each of these applications are incorporated by reference herein in its entirety.

### FIELD OF THE INVENTION

The present disclosure generally relates to tumor treatment, and particularly to methods of identifying a tumor patient for treatment with a combination of targeted therapy and immune oncology.

### BACKGROUND OF THE INVENTION

The background description includes information that may be useful in understanding the present disclosure. It is not an admission that any of the information provided herein is prior art or relevant to the presently claimed invention, or that any publication specifically or implicitly referenced is prior art.

All publications and patent applications herein are incorporated by reference to the same extent as if each individual publication or patent application were specifically and individually indicated to be incorporated by reference. Where a definition or use of a term in an incorporated reference is inconsistent or contrary to the definition of that term provided herein, the definition of that term provided herein applies and the definition of that term in the reference does not apply.

In clinical practice, tumor patients often acquire a DNA screen, usually a hotspot panel of genes. Based on this screen, if the patient does not have a targetable mutation in one of the hotspot panel of genes, they are often recommended for Immuno-Oncology (IO) therapy (e.g. IHC for PDL1). Patients that have a targetable mutation would often get on a targeted therapy. If these patients do not respond to the targeted therapy or if they relapse, then they are screened again for IO therapy. Thus a subset of tumor patients may get targeted therapy and then IO in a sequential manner.

Zhang et al have reported that the combined treatment of a patient with a CDK4/6 inhibitor (IO therapy) and a PD-1 blocker (targeted therapy) may have greater anti-tumor efficacy than treatment with each drug alone in mouse models. This combined approach has the potential to improve the treatment of patients with cancer. See Zhang et al, Cyclin D-CDK4 kinase destabilizes PD-L1 via cullin 3-SPOP to control cancer immune surveillance, Nature volume 553, pages 91-95 (4 Jan. 2018).

Similarly, Schaer et al. describe immune-modulating properties of abemaciclib, a CDK4/6 inhibitor, that include upregulation of antigen presentation on tumor cells and increased T cell activation. These activities synergize with anti-PD-L1 therapy to further enhance immune activation, including macrophage and DC antigen presentation, and also lead to a reciprocal increase in abemaciclib dependent cell cycle gene regulation. See Schaer et al, The CDK4/6 Inhibitor Abemaciclib Induces a T Cell Inflamed Tumor Microenvironment and Enhances the Efficacy of PD-L1 Checkpoint Blockade, Cell Reports 22, 2978-2994 (2018).

Li et al have disclosed PD-L1 expression was positively correlated with Fibroblast growth factor receptor 2 (FGFR2) expression in mouse model of colorectal cancer (CRC). See Li et al, FGFR2 Promotes Expression of PD-L1 in Colorectal Cancer via the JAK/STAT3 Signaling Pathway; J Immunol, Vol. 203(4) 15 Aug. 2019. The results of this study revealed a mechanism of PD-L1 expression in CRC, thus providing a theoretical basis for reversing the immune tolerance of FGFR2 overexpression in CRC.

Jiao et al have explored the crosstalk between PARP inhibition and tumor-associated immunosuppression, and provided that the combination of PARP inhibition and PD-L1 or PD-1 immune checkpoint blockade as a potential therapeutic approach to treat breast cancer. See Jiao, Shiping et al. “PARP Inhibitor Upregulates PD-L1 Expression and Enhances Cancer-Associated Immunosuppression.” Clinical cancer research: an official journal of the American Association for Cancer Research vol. 23, 14 (2017): 3711-3720.

Despite these studies, it is unclear which patients are likely to benefit from such combination treatments. It is also unclear which targeted therapy plus I0 therapy combination would result in an improved treatment. For example, Higuchi T et al reported that they did not observe any added benefit of using the anti-PD-1 and PARP inhibitor combination in an ovarian cancer mouse model. Higuchi T et al, CTLA-4 Blockade Synergizes Therapeutically with PARP Inhibition in BRCA1-Deficient Ovarian Cancer; Cancer Immunol Res. 2015 November; 3(11):1257-68. Despite differences in the dose and schedule of PD-1 antibody administration in this study, the effect of PD-1 blockade on T-cell function was not significantly different from controls.

Thus, there remains a need in the art for new methods and techniques for determining which targeted therapy and IO therapy combination would produce a synergistic effect in the treatment of cancer patients. Moreover there remains a need for identifying patients that would benefit from such combination treatment.

### SUMMARY OF THE INVENTION

The inventors have now discovered that certain tumor gene mutants are associated with differential expression of certain checkpoint inhibitors irrespective of the tissue type, and that these patterns can be used to determine if a particular patient would benefit from a combination of and IO therapy and targeted therapy that targets the gene mutants.

In one aspect, contemplated herein is a method of identifying a tumor patient for treatment with a combination of targeted therapy and immune oncology. The method disclosed comprises obtaining respective omics data, such as genomics and proteomics data for a tumor cell and a matched normal cell. The expression level of a gene transcript or gene product of a tumor sample and the matched normal sample is then determined, and the levels of expression for each of the gene transcript or gene product is compared for tumor sample and matched normal sample. Preferably, the gene transcript or gene product is a checkpoint inhibitor, selected from the group consisting of TIM3, CTLA4, TIGIT, LAG3, PDL2, PD1, IDO, OX40, PDL1, FOXP3, and/or LAG3. The matched normal sample is contemplated to be either from a healthy tissue of the same patient, or it may be from a different patient. The genomics data from the tumor sample and matched normal sample is also analyzed to determine whether the patient tumor sample has a mutation in one or more of the following genes: APC, CDKN2A, KRAS, EZH2, FBXW7, BRAF, VHL, PIK3CA, and/or MPL.

Once the genomics and proteomics data are obtained, analyzed, and calculated to see whether the mutants also differentially express checkpoints, a combination of targeted therapy and immune-oncology (IO) therapy is recommended to the patient when PD1 is overexpressed in tumor cell sample having mutated CDKN2A or mutated EZH2 gene, or when PDL1 is under-expressed in tumor cell sample having mutated APC gene, or when PDL2 is under-expressed in tumor cell sample having mutated KRAS gene, or when CTLA4 is overexpressed in tumor cell sample having mutated CDKN2A gene, or when IDO is overexpressed in tumor cell sample having mutated FBXW7 gene, or when TIM3 is under-expressed in tumor cell sample having mutated KRAS or mutated APC gene, or when LAG3 is overexpressed in tumor cell sample having mutated CDKN2A gene or mutated EZH2 gene or mutated MPL gene, or when FOXP3 is overexpressed in tumor cell sample having mutated PIK3CA gene or mutated VHL gene, or when TIGIT is overexpressed in tumor cell sample having mutated CDKN2A gene, or when OX40 is overexpressed in tumor cell sample having mutated BRAF gene.

The inventors have also found that the differential checkpoint expression patterns as disclosed herein are strongly associated with mutation status, but not driven by tissue type. Accordingly, the methods disclosed herein may be used for various types of cancer, such as thyroid cancer, brain cancer, liver cancer, prostate cancer, skin cancer, testicular cancer, kidney cancer, adrenal gland cancer, stomach cancer, pancreatic cancer, esophageal cancer, colon cancer, ovarian cancer, bladder cancer, uterus cancer, breast cancer, adipose tissue cancer, cervical cancer, lung cancer, muscle cancer, head and neck cancer, or bone marrow cancer. Furthermore, the tumor may be stomach/esophageal carcinoma, skin cutaneous melanoma, stomach adenocarcinoma, breast invasive carcinoma, or lung adenocarcinoma.

In some embodiments, the difference in expression between the patient's tumor sample and the matched normal sample is at least 10%, 20%, 30%, 40%, 50%, 60%, 70%, 80%, 90%, 100% or more. The expression level may be determined by whole genome/exome sequencing, RNA-seq, and/or proteomic analysis of the tumor, and the proteomic analysis is done via mass spectrometry.

The targeted therapy as contemplated herein comprises a therapy targeted to TIM3, CTLA4, TIGIT, LAG3, PDL2, PD1, IDO, OX40, PDL1, FOXP3, and/or LAG3. Moreover, the IO therapy comprises treatment with T-cell therapy, and/or cancer vaccines.

In another aspect, disclosed herein is a method of identifying correlations between specific gene mutations and expression of checkpoint inhibitors, and using the correlations to prepare a combination treatment for a tumor patient. The method comprises obtaining respective omics data for a tumor cell sample and a matched normal cell sample. The proteomics data in these omics samples are used to determine the expression level of an immune checkpoint inhibitor selected from the group consisting of TIM3, CTLA4, TIGIT, LAG3, PDL2, PD1, IDO, OX40, PDL1, FOXP3, and/or LAG3 in the tumor sample and comparing to the corresponding expression level in a matched normal sample. The genomics data in the samples are used for determining that the patient has a mutation in a gene selected from the group comprising APC, CDKN2A, KRAS, EZH2, FBXW7, BRAF, VHL, PIK3CA, and/or MPL. Then, the patient is treat with a combination of targeted therapy and IO therapy when one of the following correlations are identified: (a) PD1 is overexpressed in tumor cell sample having mutated CDKN2A or mutated EZH2 gene, or (b) when PDL1 is under-expressed in tumor cell sample having mutated APC gene, or (c) PDL2 is under-expressed in tumor cell sample having mutated KRAS gene; or (d) CTLA4 is overexpressed in tumor cell sample having mutated CDKN2A gene; or (e) IDO is overexpressed in tumor cell sample having mutated FBXW7 gene; or (f) TIM3 is under-expressed in tumor cell sample having mutated KRAS or mutated APC gene, or (g) LAG3 is overexpressed in tumor cell sample having mutated CDKN2A gene or mutated EZH2 gene or mutated MPL gene, or (h) FOXP3 is overexpressed in tumor cell sample having mutated PIK3CA gene or mutated VHL gene; or (i) TIGIT is overexpressed in tumor cell sample having mutated CDKN2A gene; or (j) OX40 is overexpressed in tumor cell sample having mutated BRAF gene.

In another aspect, disclosed herein is a method of treating a patient having a tumor, comprising: obtaining genomics and transcriptomics data from a tumor sample of the patient and a matched normal sample; determining the expression level, in the tumor sample and matched normal sample, of a checkpoint inhibitor selected from the group consisting of TIM3, CTLA4, TIGIT, LAG3, PDL2, PD1, IDO, OX40, PDL1, FOXP3, and/or LAG3; determining that the tumor sample has a mutation in a gene selected from the group comprising APC, CDKN2A, KRAS, EZH2, FBXW7, BRAF, VHL, PIK3CA, and/or MPL; and treating the patient by administering a combination of (i) targeted therapy and (ii) immune-oncology (IO) therapy to the patient, upon determination that (a) PD1 is overexpressed in tumor cell sample having mutated CDKN2A or mutated EZH2 gene, or (b) when PDL1 is under-expressed in tumor cell sample having mutated APC gene, or (c) PDL2 is under-expressed in tumor cell sample having mutated KRAS gene; or (d) CTLA4 is overexpressed in tumor cell sample having mutated CDKN2A gene; or (e) IDO is overexpressed in tumor cell sample having mutated FBXW7 gene; or (f) TIM3 is under-expressed in tumor cell sample having mutated KRAS or mutated APC gene, or (g) LAG3 is overexpressed in tumor cell sample having mutated CDKN2A gene or mutated EZH2 gene or mutated MPL gene, or (h) FOXP3 is overexpressed in tumor cell sample having mutated PIK3CA gene or mutated VHL gene; or (i) TIGIT is overexpressed in tumor cell sample having mutated CDKN2A gene; or (j) OX40 is overexpressed in tumor cell sample having mutated BRAF gene.

Various objects, features, aspects, and advantages will become more apparent from the following detailed description of preferred embodiments, along with the accompanying drawing in which like numerals represent like components.

### BRIEF DESCRIPTION OF THE DRAWINGS

FIGS. 1A and 1B depicts an exemplary embodiment of the datasets used in one study disclosed herein.

FIGS. 2A, 2B, 2C, and 2D depicts an exemplary embodiment of significantly differentially expressed checkpoints in presence of mutants.

FIG. 3 depicts an exemplary embodiment of genes with at least one significant differential checkpoint expression pattern.

FIG. 4A-4J depicts an exemplary embodiment of distributions for 15 tissue-independent mutation-associated checkpoint differentiators.

FIG. 5 depicts an exemplary embodiment of genes with at least one significant differential checkpoint expression pattern.

### DETAILED DESCRIPTION

In clinical practice, cancer patients often get a DNA screen of some sort done for a hotspot panel of genes before treatment with a targeted therapy or IO therapy. Patients that do not have a targetable mutation, usually receive a screening for an IO therapy (e.g. IHC for PDL1). Patients that do have a targetable mutation would often get on a targeted therapy, then not respond or relapse and so will also get screened for IO therapy later. Moreover, combination drug trials (e.g. Parpinhibitor+pembro, CDK4/6 inhibitor+ipi, etc) are also being run. However, even with this progress, currently there is no resource to know which targeted therapy in combination with which IO therapy are likely good as combinations for a particular patient. Moreover, it is not currently known whether a patient would have a synergistic effect from taking a combination of targeted therapy and IO therapy, or whether these two therapies should be explored serially.

The inventors have found a solution to this problem and noticed that patients that have targetable mutations are often the ones that result in having a positive marker for IO therapy, but the ones without targetable mutations often do not have markers for IO therapy. Furthermore, the inventors have disclosed herein that differential checkpoint expression patterns are strongly associated with mutation status, and are not primarily driven by tissue type. As disclosed herein, patients having these differential checkpoint expression patterns are contemplated to have a synergistic effect in tumor treatment when the targeted therapy and IO therapy are combined and administered together.

As used herein, the term “tumor” refers to, and is interchangeably used with one or more cancer, cancer cells, cancer tissues, malignant tumor cells, or malignant tumor tissue, that can be placed or found in one or more anatomical locations in a human body. It should be noted that the term “patient” as used herein includes both individuals that are diagnosed with a condition (e.g., cancer) as well as individuals undergoing examination and/or testing for the purpose of detecting or identifying a condition. Thus, a patient having a tumor refers to both individuals that are diagnosed with a cancer as well as individuals that are suspected to have a cancer. Immunologically “cold” tumors are cancers that for various reasons contain few infiltrating T cells and are not recognized and do not provoke a strong response by the immune system. Cancers that are classically immunologically cold include glioblastomas as well as ovarian, prostate, pancreatic, and most breast cancers. In contrast, immunologically “hot” tumors contain high levels of infiltrating T cells and more antigens, making them more recognizable by the immune system and more likely to trigger a strong immune response. Among the cancers considered to be immunologically hot are bladder, head and neck, kidney, melanoma, and non-small cell lung cancers. As used herein, the term “provide” or “providing” refers to and includes any acts of manufacturing, generating, placing, enabling to use, transferring, or making ready to use.

In one aspect, the instant disclosure provides a method of identifying a tumor patient for treatment with a combination of targeted therapy and immune oncology (IO). Targeted cancer therapies are drugs or other substances that block the growth and spread of cancer by interfering with specific molecules (“molecular targets”) that are involved in the growth, progression, and spread of cancer. For example, a targeted therapy could reduce the activity of the target or prevent it from binding to a receptor that it normally activates, among other possible mechanisms. Most targeted therapies are either small molecules or monoclonal antibodies. On the other hand, IO therapies contemplated herein treats cancer by using the power of the body's own immune system to prevent, control, and eliminate cancer. Immunotherapy of IO may educate the immune system to recognize and attack specific cancer cells, boost immune cells to help them eliminate cancer, and provide the body with additional components to enhance the immune response. Examples of cancer immunotherapy contemplated herein comprise T cell therapy, cancer vaccines, and adoptive cell transfer.

### Obtaining Omics Data

The disclosure provided herein comprises methods for obtaining omics data from a tumor cell and a matched normal cell. Any suitable methods and/or procedures to obtain omics data are contemplated. For example, the omics data can be obtained by obtaining tissues from an individual and processing the tissue to obtain DNA, RNA, protein, or any other biological substances from the tissue to further analyze relevant information. In another example, the omics data can be obtained directly from a database that stores omics information of an individual.

Where the omics data is obtained from the tissue of an individual, any suitable methods of obtaining a tumor sample (tumor cells or tumor tissue) or normal (or healthy) tissue from the patient are contemplated. Most typically, a tumor sample or normal tissue sample can be obtained from the patient via a biopsy (including liquid biopsy, or obtained via tissue excision during a surgery or an independent biopsy procedure, etc.), which can be fresh or processed (e.g., frozen, etc.) until further process for obtaining omics data from the tissue. For example, tissues or cells may be fresh or frozen. In other example, the tissues or cells may be in a form of cell/tissue extracts. In some embodiments, the tissues or cells may be obtained from a single or multiple different tissues or anatomical regions. For example, a metastatic breast cancer tissue can be obtained from the patient's breast as well as other organs (e.g., liver, brain, lymph node, blood, lung, etc.) for metastasized breast cancer tissues. In another example, a normal tissue or matched normal tissue (e.g., patient's non-cancerous breast tissue) of the patient can be obtained from any part of the body or organs, preferably from liver, blood, or any other tissues near the tumor (in a close anatomical distance, etc.).

In some embodiments, tumor samples can be obtained from the patient in multiple time points in order to determine any changes in the tumor samples over a relevant time period. For example, tumor samples (or suspected tumor samples) may be obtained before and after the samples are determined or diagnosed as cancerous. In another example, tumor samples (or suspected tumor samples) may be obtained before, during, and/or after (e.g., upon completion, etc.) a one time or a series of a cancer treatment (e.g., radiotherapy, chemotherapy, immunotherapy, etc.). In still another example, the tumor samples (or suspected tumor samples) may be obtained during the progress of the tumor upon identifying a new metastasized tissues or cells.

From the obtained tumor samples (cells or tissue) or healthy samples (cells or tissue), DNA (e.g., genomic DNA, extrachromosomal DNA, etc.), RNA (e.g., mRNA, miRNA, siRNA, shRNA, etc.), and/or proteins (e.g., membrane protein, cytosolic protein, nucleic protein, etc.) can be isolated and further analyzed to obtain omics data. Alternatively and/or additionally, a step of obtaining omics data may include receiving omics data from a database that stores omics information of one or more patients and/or healthy individuals. For example, omics data of the patient's tumor may be obtained from isolated DNA, RNA, and/or proteins from the patient's tumor tissue, and the obtained omics data may be stored in a database (e.g., cloud database, a server, etc.) with other omics data set of other patients having the same type of tumor or different types of tumor. Omics data obtained from the healthy individual or the matched normal tissue (or normal tissue) of the patient can be also stored in the database such that the relevant data set can be retrieved from the database upon analysis. Likewise, where protein data are obtained, these data may also include protein activity, especially where the protein has enzymatic activity (e.g., polymerase, kinase, hydrolase, lyase, ligase, oxidoreductase, etc.).

As used herein, omics data includes but is not limited to information related to genomics, proteomics, and transcriptomics, as well as specific gene expression or transcript analysis, and other characteristics and biological functions of a cell. With respect to genomics data, suitable genomics data includes DNA sequence analysis information that can be obtained by whole genome sequencing and/or exome sequencing (typically at a coverage depth of at least 10×, more typically at least 20×) of both tumor and matched normal sample. Alternatively, DNA data may also be provided from an already established sequence record (e.g., SAM, BAM, FASTA, FASTQ, or VCF file) from a prior sequence determination. Therefore, data sets may include unprocessed or processed data sets, and exemplary data sets include those having BAM format, SAM format, FASTQ format, or FASTA format. However, it is especially preferred that the data sets are provided in BAM format or as BAMBAM diff objects (e.g., US2012/0059670A1 and US2012/0066001A1). Omics data can be derived from whole genome sequencing, exome sequencing, transcriptome sequencing (e.g., RNA-seq), or from gene specific analyses (e.g., PCR, qPCR, hybridization, LCR, etc.). Likewise, computational analysis of the sequence data may be performed in numerous manners. In most preferred methods, however, analysis is performed in silico by location-guided synchronous alignment of tumor and normal samples as, for example, disclosed in US 2012/0059670A1 and US 2012/0066001A1 using BAM files and BAM servers. Such analysis advantageously reduces false positive neoepitopes and significantly reduces demands on memory and computational resources.

Where it is desired to obtain the tumor-specific omics data, numerous manners are deemed suitable for use herein so long as such methods will be able to generate a differential sequence object or other identification of location-specific difference between tumor and matched normal sequences. Exemplary methods include sequence comparison against an external reference sequence (e.g., hg18, or hg19), sequence comparison against an internal reference sequence (e.g., matched normal), and sequence processing against known common mutational patterns (e.g., SNVs). Therefore, contemplated methods and programs to detect mutations between tumor and matched normal, tumor and liquid biopsy, and matched normal and liquid biopsy include iCallSV (URL: github.com/rhshah/iCallSV), VarScan (URL: varscan.sourceforge.net), MuTect (URL: github.com/broadinstitute/mutect), Strelka (URL: github.com/Illumina/strelka), Somatic Sniper (URL: gmt.genome.wustl.edu/somatic-sniper/), and BAMBAM (US 2012/0059670).

However, in especially preferred aspects of the inventive subject matter, the sequence analysis is performed by incremental synchronous alignment of the first sequence data (tumor sample) with the second sequence data (matched normal), for example, using an algorithm as for example, described in Cancer Res 2013 Oct. 1; 73(19):6036-45, US 2012/0059670 and US 2012/0066001 to so generate the patient and tumor specific mutation data. As will be readily appreciated, the sequence analysis may also be performed in such methods comparing omics data from the tumor sample and matched normal omics data to so arrive at an analysis that can not only inform a user of mutations that are genuine to the tumor within a patient, but also of mutations that have newly arisen during treatment (e.g., via comparison of matched normal and matched normal/tumor, or via comparison of tumor). In addition, using such algorithms (and especially BAMBAM), allele frequencies and/or clonal populations for specific mutations can be readily determined, which may advantageously provide an indication of treatment success with respect to a specific tumor cell fraction or population. Thus, exemplary subtypes of genomics data may include, but not limited to genome amplification (as represented genomic copy number aberrations), somatic mutations (e.g., point mutation (e.g., nonsense mutation, missense mutation, etc.), deletion, insertion, etc.), genomic rearrangements (e.g., intrachromosomal rearrangement, extrachromosomal rearrangement, translocation, etc.), appearance and copy numbers of extrachromosomal genomes (e.g., double minute chromosome, etc.). In addition, genomic data may also include mutation burden that is measured by the number of mutations carried by the cells or appeared in the cells in the tissue in a predetermined period of time or within a relevant time period.

Moreover, it should be noted that some data sets are preferably reflective of a tumor and a matched normal sample of the same patient to so obtain patient and tumor specific information. In such embodiments, genetic germ line alterations not giving rise to the tumor (e.g., silent mutation, SNP, etc.) can be excluded. Of course, it should be recognized that the tumor sample may be from an initial tumor, from the tumor upon start of treatment, from a recurrent tumor or metastatic site, etc. In most cases, the matched normal sample of the patient may be blood, or non-diseased tissue from the same tissue type as the tumor.

In addition, omics data of cancer and/or normal cells comprises transcriptome data set that includes sequence information and expression level (including expression profiling, copy number, or splice variant analysis) of RNA(s) (preferably cellular mRNAs) that is obtained from the patient, from the cancer tissue (diseased tissue) and/or matched normal tissue of the patient or a healthy individual. There are numerous methods of transcriptomic analysis known in the art, and all of the known methods are deemed suitable for use herein (e.g., RNAseq, RNA hybridization arrays, qPCR, etc.). Consequently, preferred materials include mRNA and primary transcripts (hnRNA), and RNA sequence information may be obtained from reverse transcribed polyA⁺-RNA, which is in turn obtained from a tumor sample and a matched normal (healthy) sample of the same patient. Likewise, it should be noted that while polyA⁺-RNA is typically preferred as a representation of the transcriptome, other forms of RNA (hn-RNA, non-polyadenylated RNA, siRNA, miRNA, etc.) are also deemed suitable for use herein. Preferred methods include quantitative RNA (hnRNA or mRNA) analysis and/or quantitative proteomics analysis, especially including RNAseq. In other aspects, RNA quantification and sequencing is performed using RNA-seq, qPCR and/or rtPCR based methods, although various alternative methods (e.g., solid phase hybridization-based methods) are also deemed suitable. Viewed from another perspective, transcriptomic analysis may be suitable (alone or in combination with genomic analysis) to identify and quantify genes having a cancer- and patient-specific mutation.

Preferably, the transcriptomics data set includes allele-specific sequence information and copy number information. In such embodiment, the transcriptomics data set includes all read information of at least a portion of a gene, preferably at least 10×, at least 20×, or at least 30×. Allele-specific copy numbers, more specifically, majority and minority copy numbers, are calculated using a dynamic windowing approach that expands and contracts the window's genomic width according to the coverage in the germline data, as described in detail in U.S. Pat. No. 9,824,181, which is incorporated by reference herein. As used herein, the majority allele is the allele that has majority copy numbers (>50% of total copy numbers (read support) or most copy numbers) and the minority allele is the allele that has minority copy numbers (<50% of total copy numbers (read support) or least copy numbers).

It should be appreciated that one or more desired nucleic acids or genes may be selected for a particular disease (e.g., cancer, etc.), disease stage, specific mutation, or even on the basis of personal mutational profiles or presence of expressed neoepitopes. Alternatively, where discovery or scanning for new mutations or changes in expression of a particular gene is desired, RNAseq is preferred to so cover at least part of a patient transcriptome. Moreover, it should be appreciated that analysis can be performed static or over a time course with repeated sampling to obtain a dynamic picture without the need for biopsy of the tumor or a metastasis.

Further, omics data of cancer and/or normal cells comprises proteomics data set that includes protein expression levels (quantification of protein molecules), post-translational modification, protein-protein interaction, protein-nucleotide interaction, protein-lipid interaction, and so on. Thus, it should also be appreciated that proteomic analysis as presented herein may also include activity determination of selected proteins. Such proteomic analysis can be performed from freshly resected tissue, from frozen or otherwise preserved tissue, and even from FFPE tissue samples. Most preferably, proteomics analysis is quantitative (i.e., provides quantitative information of the expressed polypeptide) and qualitative (i.e., provides numeric or qualitative specified activity of the polypeptide). Any suitable types of analysis are contemplated. However, particularly preferred proteomics methods include antibody-based methods and mass spectroscopic methods. Moreover, it should be noted that the proteomics analysis may not only provide qualitative or quantitative information about the protein per se, but may also include protein activity data where the protein has catalytic or other functional activity. One exemplary technique for conducting proteomic assays is described in U.S. Pat. No. 7,473,532, incorporated by reference herein. Further suitable methods of identification and even quantification of protein expression include various mass spectroscopic analyses (e.g., selective reaction monitoring (SRM), multiple reaction monitoring (MRM), and consecutive reaction monitoring (CRM)).

The expression level of a gene transcript or gene product of a tumor sample and the matched normal sample is then determined, and the levels of expression for each of the gene transcript or gene product is compared for tumor sample and matched normal sample. The matched normal sample is contemplated to be either from a healthy tissue of the same patient, or it may be from a different patient. The genomics data from the tumor sample and matched normal sample is also analyzed to determine whether the patient tumor sample has a mutation in one or more genes. Once the genomics and proteomics data are obtained, analyzed, and calculated to see whether the mutants also differentially express checkpoints. A combination of targeted therapy and immune-oncology (IO) therapy is recommended to the patient when PD1 is overexpressed in tumor cell sample having mutated CDKN2A or mutated EZH2 gene, or when PDL1 is under-expressed in tumor cell sample having mutated APC gene, or when PDL2 is under-expressed in tumor cell sample having mutated KRAS gene; or when CTLA4 is overexpressed in tumor cell sample having mutated CDKN2A gene; or when IDO is overexpressed in tumor cell sample having mutated FBXW7 gene; or when TIM3 is under-expressed in tumor cell sample having mutated KRAS or mutated APC gene, or when LAG3 is overexpressed in tumor cell sample having mutated CDKN2A gene or mutated EZH2 gene or mutated MPL gene, or when FOXP3 is overexpressed in tumor cell sample having mutated PIK3CA gene or mutated VHL gene; or when TIGIT is overexpressed in tumor cell sample having mutated CDKN2A gene; or when OX40 is overexpressed in tumor cell sample having mutated BRAF gene.

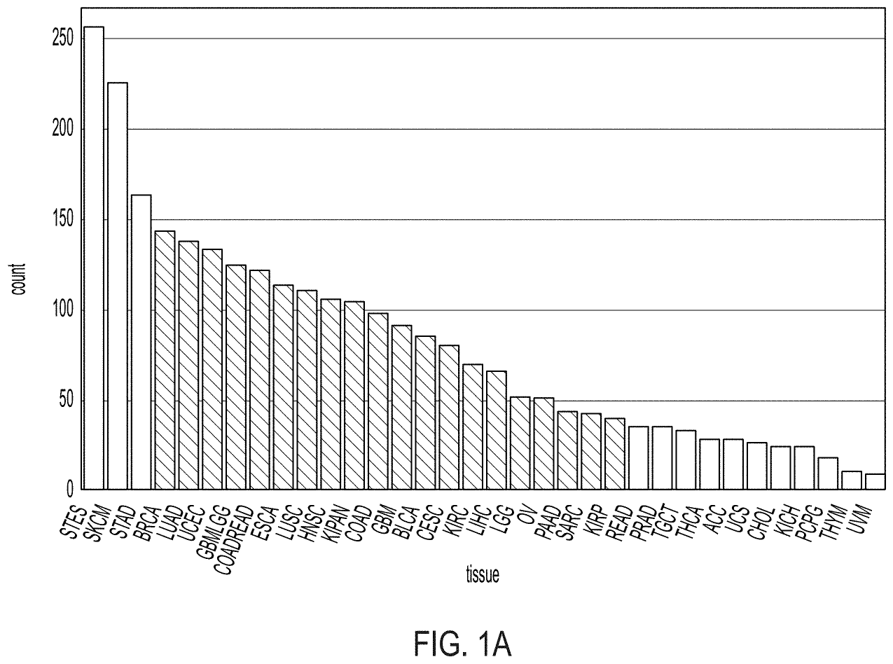

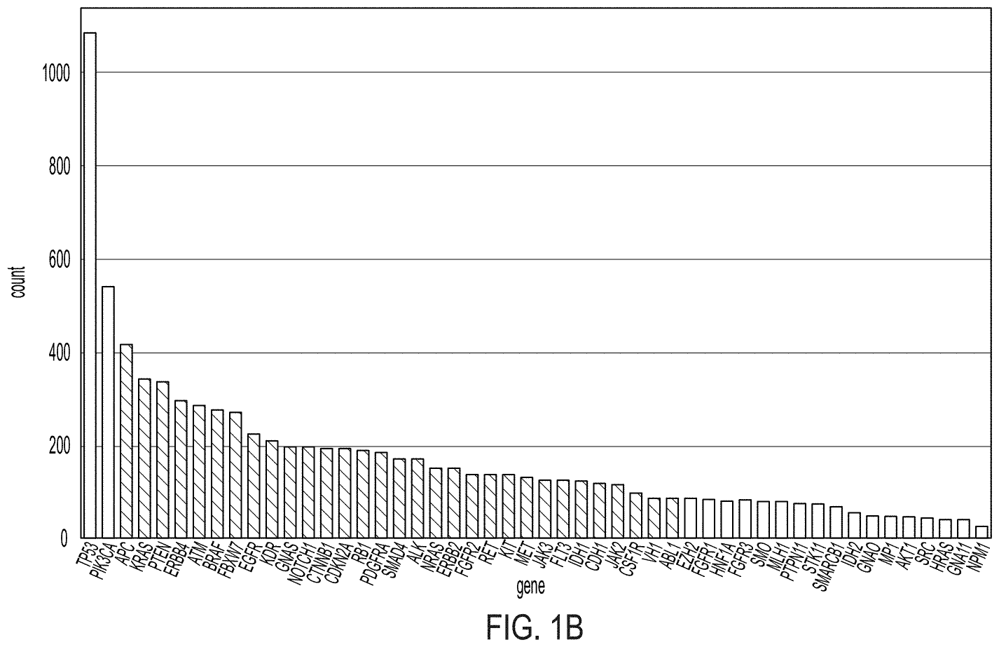

In one illustrative embodiment, as illustrated in FIG. 1A and FIG. 1B the inventor obtained TCGA somatic mutation calls from Broad's firehose for 9037 exomes across 36 ‘tissues’ (here lung adeno and squamous are considered different tissues). 2805 patients were identified with at least one non-synonymous SNV or indel in one of the 50 ampliseq hotspot genes. The inventor then removed any of LAME, DLBC, MESO, or LCML (i.e. non-solid) from the dataset. The analysis dataset was thus arrived at 2740 patients.

To see if mutants also differentially express checkpoints, the inventor performed t-tests on expression of key checkpoint transcripts (PD1, PDL1, PDL2, CTLA4, TIGIT, IDO, OX40, FOXP3, LAG3, TIM3) between mutant and wild type for each hotspot gene. Only those comparisons that passed p<0.05 after Bonferroni adjustment was kept for further analysis.

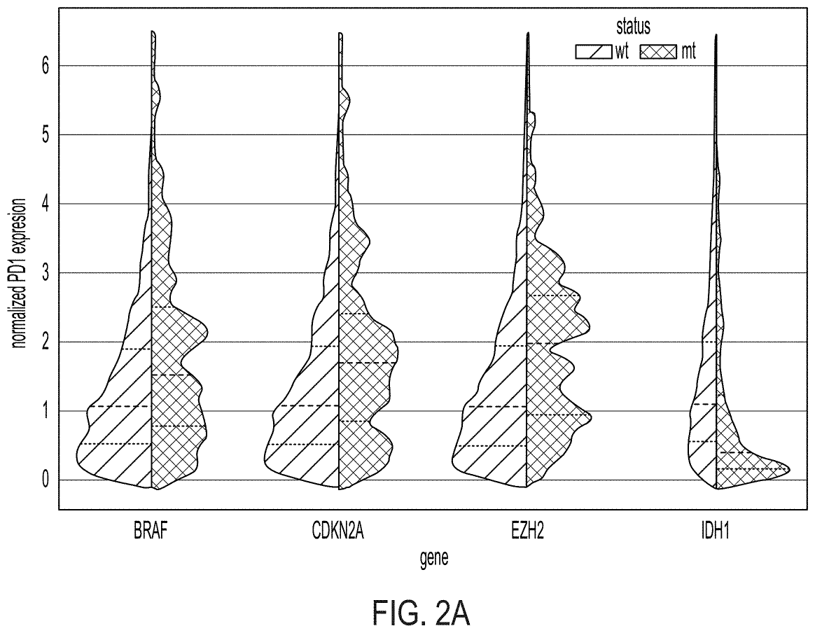

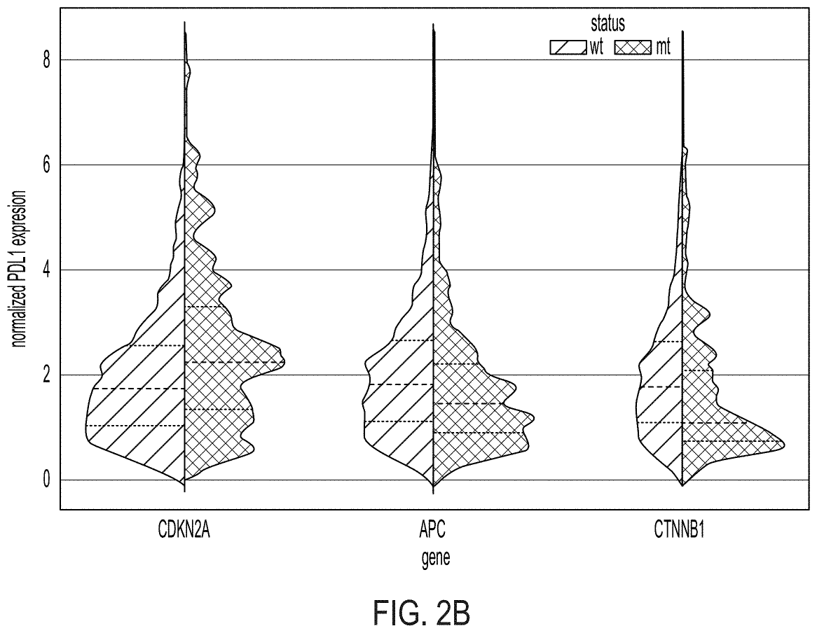

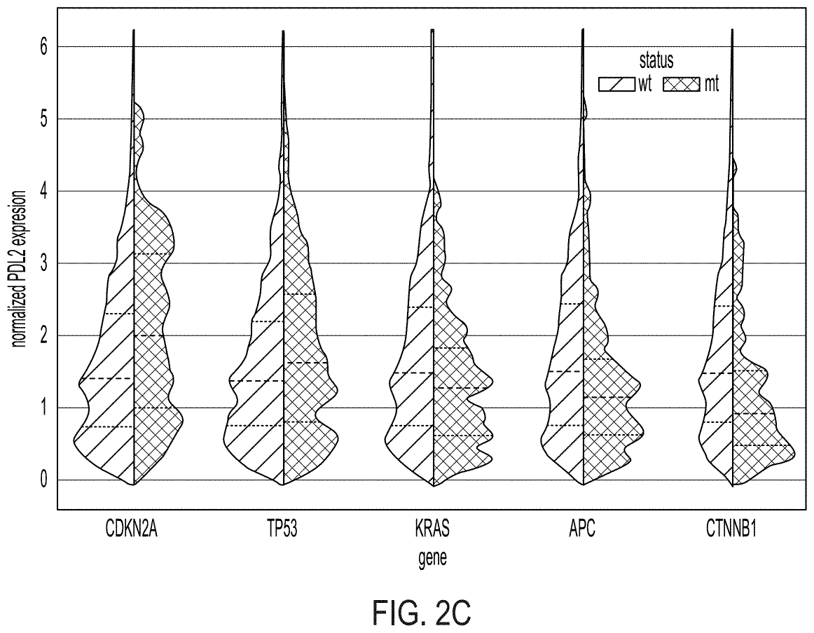

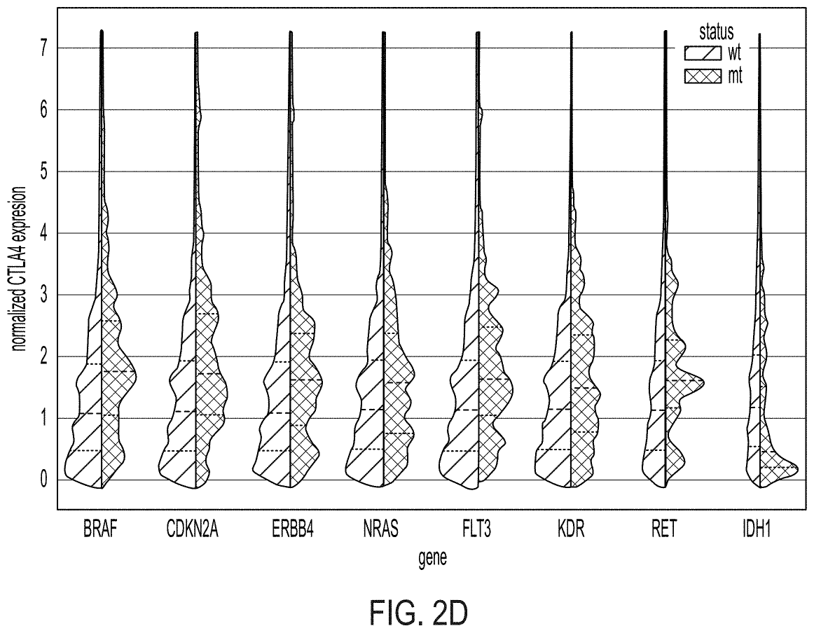

To ensure tissue was not confounding observed differential expression patterns, the inventor identified the tissue most enriched for mutants in each gene by one-sided Fisher's exact test. Then, similar t-tests were performed between most-enriched-tissue vs. other tissues for each checkpoint differential expression. Only those differential checkpoint patterns that were more associated with mutation status than most-enriched-tissue were kept. As illustrated in FIGS. 2-6, the resulting presented differential checkpoint expression patterns are strongly associated with mutation status, and are not primarily driven by tissue type.

FIG. 2A-D illustrates examples of significantly differentially expressed checkpoints in presence of mutants. The normalized expression of PD1, PDL1/2, and CTLA4 are shown in FIG. 2A-D. The y-axis is normalized expression (log 2[TPM+1]), while the x-axis is each gene that was found to significantly affect checkpoint expression. Each blue portion is the distribution of checkpoint expression in wild type gene, and green is in mutant gene, for each gene marked on the x-axis. Quartiles are marked in dotted lines. This demonstrates that there is very marked differential expression between mutant type and wild type for some of these genes. There were 46 such significant associations (20 shown in FIG. 2A-D).

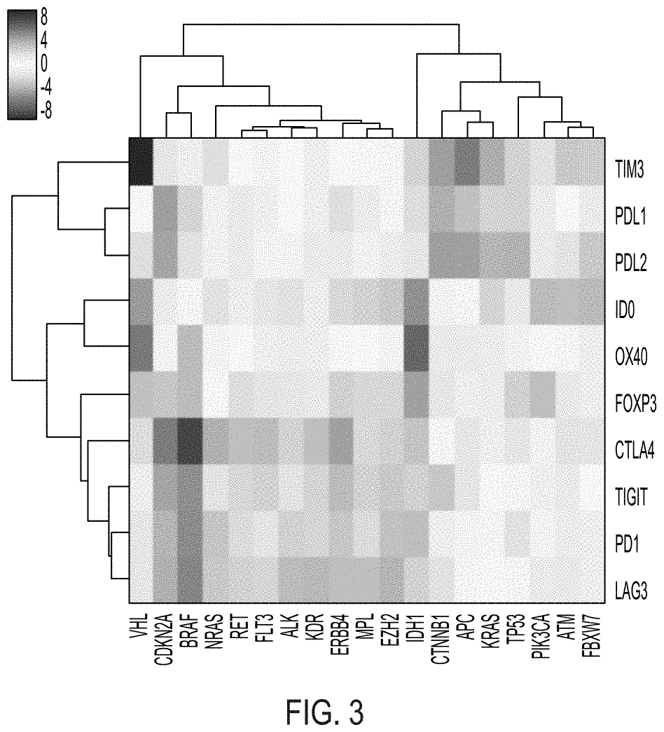

FIG. 3 illustrates genes with at least one significant differential checkpoint expression pattern before removing those confounded by tissue. X-axis is mutant genes, y-axis is checkpoint markers, each cell is colored by t-statistic for differential expression (blue=higher in wild type gene, red=higher in mutant type gene). In one embodiment, this is effectively the difference between blue and green distributions shown in FIG. 2, but for all checkpoints. Both axes in FIG. 3 are organized by hierarchical clustering.

The overall “redness” in FIG. 3 illustrates that checkpoints expression is positively associated with mutants in many of these genes. The blue at the top-right suggests mutants in Wnt (CTNNB1, APC) and compensatory Akt pathways (PIK3CA, KRAS) are immunosuppressive similarly to PDL1/2 expression, as checkpoints are higher in wild type than mutant type samples. Some genes that are very hot (like BRAF) are known to be associated with immune-hot tissues like melanoma, and so may not be driven by mutation status alone.

Table 1 illustrates the most statistically-enriched tissue for mutants in each hotspot gene, sorted by p-value. P-values for enrichment were calculated from the Odds Ratio (OR) using Fisher's exact test.

TABLE 1

| | tissue | OR | p |
|---|---|---|---|
| BRAF | SKCM | 12.6 | 6.91E−54 |
| PTEN | UCEC | 19.1 | 4.54E−53 |
| NRAS | SKCM | 14.6 | 5.71E−42 |
| APC | COADREAD | 12.1 | 7.61E−37 |
| VHL | KIRC | 39 | 9.29E−31 |
| KRAS | PAAD | 20.8 | 1.25E−20 |
| IDH1 | LGG | 23.4 | 3.04E−20 |
| PIK3CA | UCEC | 5 | 6.13E−18 |
| STK11 | LUAD | 13.7 | 9.21E−17 |
| TP53 | ESCA | 4.7 | 1.45E−14 |
| EGFR | GBM | 6 | 1.71E−11 |
| FGFR2 | UCEC | 6 | 5.12E−11 |
| CTNNB1 | UCEC | 4.8 | 1.55E−10 |
| NOTCH1 | HNSC | 5.3 | 3.28E−10 |
| KDR | SKCM | 3.5 | 6.62 E−10 |
| FBXW7 | COADREAD | 4.2 | 9.29E−10 |
| GNAQ | UVM | 103.5 | 2.79E−09 |
| CDKN2A | HNSC | 4.9 | 4.30E−09 |
| CDH1 | BRCA | 5.3 | 6.10E−09 |
| GNA11 | UVM | 81.7 | 9.38E−08 |
| ERBB4 | SKCM | 2.6 | 3.75E−07 |
| SMAD4 | COADREAD | 4 | 6.25E−07 |
| ALK | SKCM | 3 | 8.73E−07 |
| FLT3 | SKCM | 3.1 | 9.28E−06 |
| FGFR3 | BLCA | 5.7 | 2.13E−05 |
| MET | SKCM | 2.9 | 3.01E−05 |
| GNAS | STES | 2.3 | 4.22E−05 |
| PDGFRA | SKCM | 2.5 | 5.91E−05 |
| KIT | UCEC | 3.1 | 0.00016599 |
| EZH2 | UCEC | 3.7 | 0.00039408 |
| ATM | UCEC | 2.2 | 0.00079409 |
| RB1 | GBM | 2.9 | 0.00089374 |
| HRAS | BLCA | 6.1 | 0.00098822 |
| SMO | STES | 2.7 | 0.0014682 |
| FGFR1 | STAD | 3 | 0.00203827 |
| JAK2 | UCEC | 2.8 | 0.00227983 |
| CSF1R | STES | 2.4 | 0.00229188 |
| IDH2 | LGG | 6 | 0.00267961 |
| HNF1A | SKCM | 2.5 | 0.00436086 |
| MPL | UCEC | 3.8 | 0.00479974 |
| ABL1 | SKCM | 2.4 | 0.00616198 |
| ERBB2 | STES | 1.9 | 0.00775635 |
| SMARCB1 | STAD | 2.7 | 0.0109901 |
| NPM1 | LUAD | 4.5 | 0.0193403 |
| JAK3 | STES | 1.7 | 0.0329572 |
| SRC | BLCA | 3.4 | 0.0408976 |
| RET | SKCM | 1.7 | 0.0430112 |
| PTPN11 | UCEC | 2.2 | 0.0595462 |
| AKT1 | THCA | 4.8 | 0.0747864 |
| MLH1 | COADREAD | 1.9 | 0.108947 |

Table 2 illustrates mutation-associated differential checkpoint expression patterns that exceed tissue effects. The tissue most enriched for mutations in these genes was identified, and a t-test was performed using the enriched tissue to split instead of mutation status. 15/44 significant differential checkpoint patterns were more differential between wild type and mutants than between the most mutant-associated tissue vs. others (i.e. adj. p<tissue_adj_p). This set of mutation-associated checkpoints are likely not-confounded by tissue, but are rather strongly associated with mutation status. This list is suggestive of targeted therapies+IO targets to prioritize, regardless of tissue type.

TABLE 2

| | | | | | enriched_ | | | | mt_beats_ |
|---|---|---|---|---|---|---|---|---|---|
| ict_gene | mt_gene | t | p | adj. p | tissue | tissue_t | tissue_p | tissue_adj_p | tissue |
| TIM3 | APC | −7.215214 | 7.55E−13 | 3.77E−10 | COADREAD | −5.60739 | 2.33E−08 | 1.07E−06 | TRUE |
| CTLA4 | CDKN2A | 6.984055 | 3.86E−12 | 1.93E−09 | HNSC | 5.36374 | 9.07E−08 | 4.17E−06 | TRUE |
| TIGIT | CDKN2A | 5.205507 | 2.13E−07 | 1.06E−04 | HNSC | 4.45401 | 8.88E−06 | 0.0004086 | TRUE |
| TIM3 | KRAS | −5.020813 | 5.59E−07 | 2.80E−04 | PAAD | 0.780041 | 0.435457 | 1 | TRUE |
| LAG3 | CDKN2A | 4.785373 | 1.83E−06 | 9.14E−04 | HNSC | 2.13464 | 0.0329092 | 1 | TRUE |
| PDL2 | KRAS | −4.697771 | 2.80E−06 | 1.40E−03 | PAAD | −0.921901 | 0.356689 | 1 | TRUE |
| LAG3 | EZH2 | 4.581267 | 4.90E−06 | 2.45E−03 | UCEC | 2.61226 | 0.0090604 | 0.41678 | TRUE |
| PD1 | CDKN2A | 4.551732 | 5.63E−06 | 2.82E−03 | HNSC | 0.719399 | 0.471977 | 1 | TRUE |
| IDO | FBXW7 | 4.34461 | 1.46E−05 | 7.32E−03 | COADREAD | 0.928691 | 0.353159 | 1 | TRUE |
| OX40 | BRAF | 4.212699 | 2.63E−05 | 1.32E−02 | SKCM | 2.32015 | 0.0204305 | 0.939803 | TRUE |
| PDL1 | APC | −4.091382 | 4.45E−05 | 2.23E−02 | COADREAD | −3.23998 | 0.0012145 | 0.0558685 | TRUE |
| FOXP3 | VHL | −4.078441 | 4.71E−05 | 2.35E−02 | KIRC | −1.98323 | 0.0474752 | 1 | TRUE |
| FOXP3 | PIK3CA | 4.034301 | 5.68E−05 | 2.84E−02 | UCEC | 2.65184 | 0.0080673 | 0.371094 | TRUE |
| LAG3 | MPL | 3.945571 | 8.23E−05 | 4.11E−02 | UCEC | 2.61226 | 0.0090604 | 0.41678 | TRUE |
| PD1 | EZH2 | 3.932862 | 8.67E−05 | 4.34E−02 | UCEC | 2.66792 | 0.0076924 | 0.353848 | TRUE |

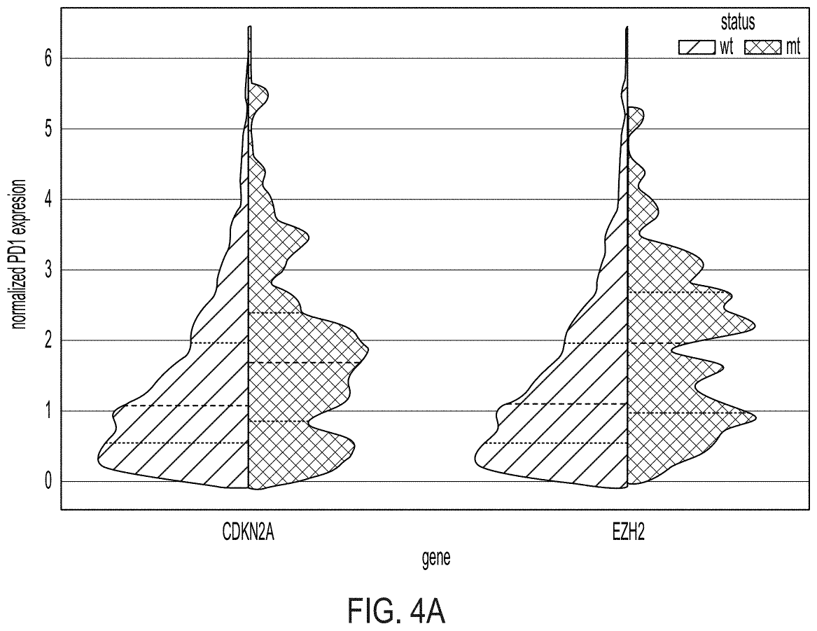

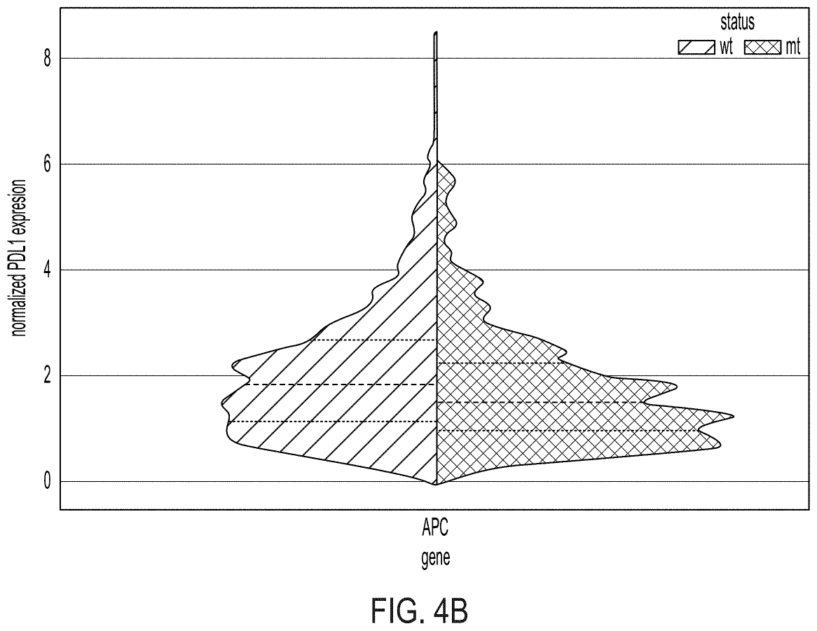

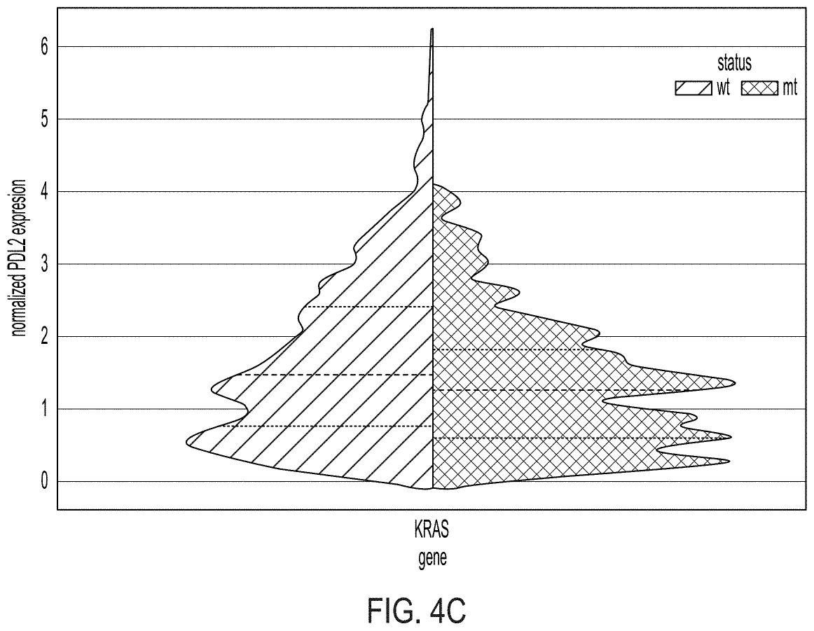

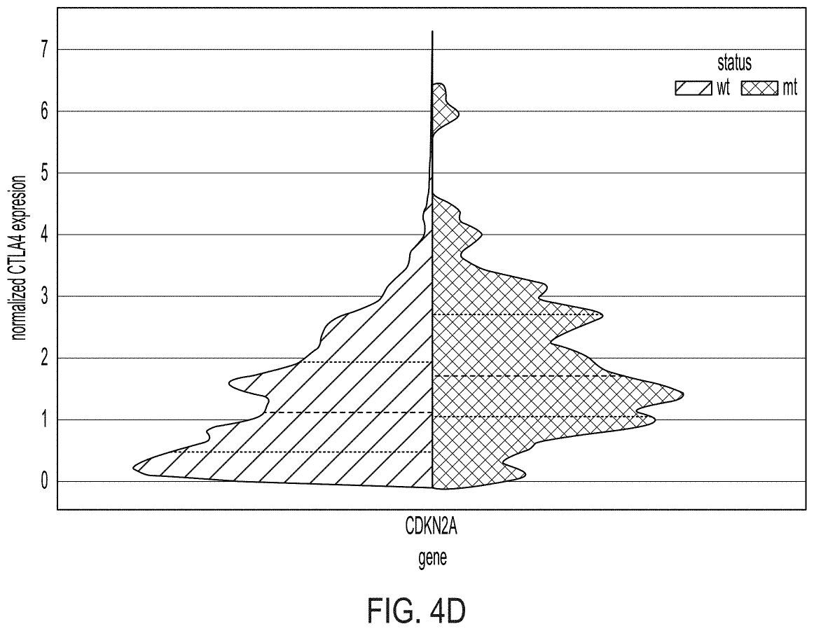

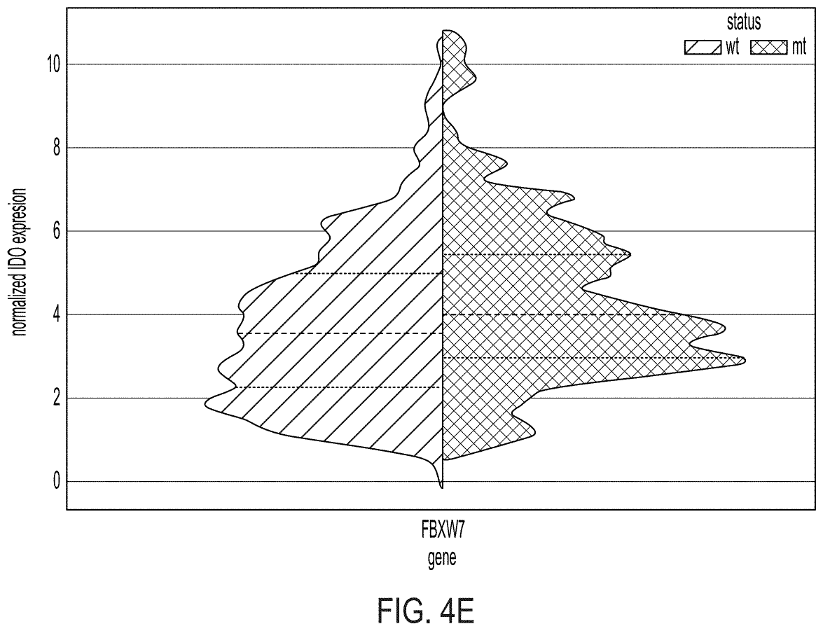

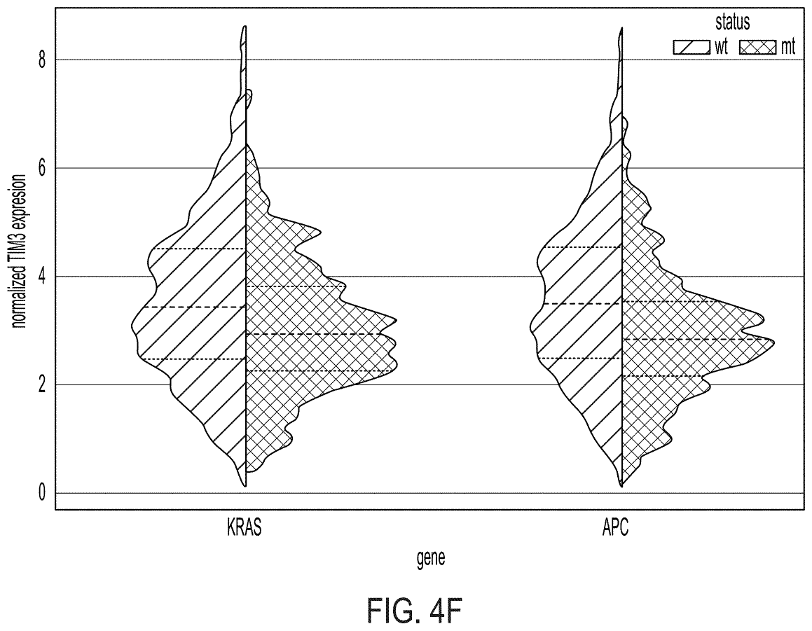

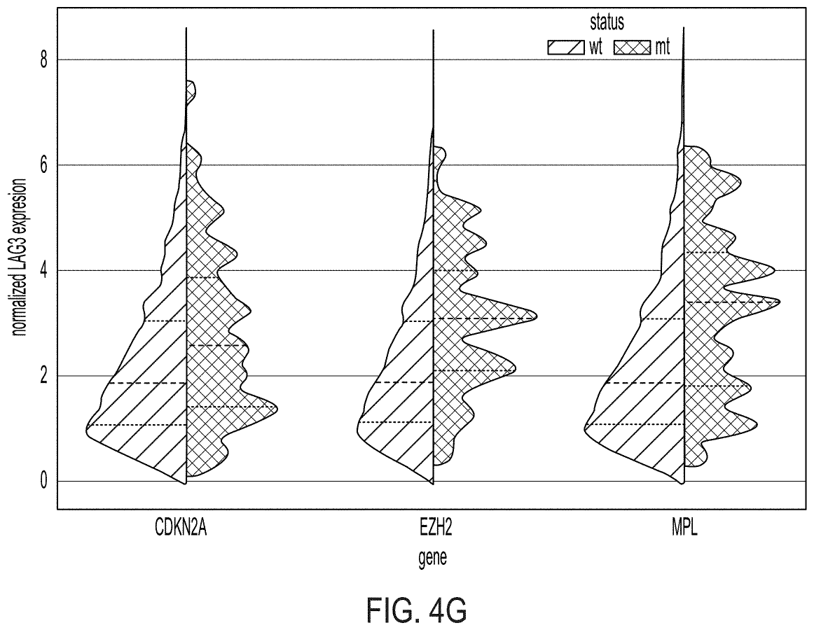

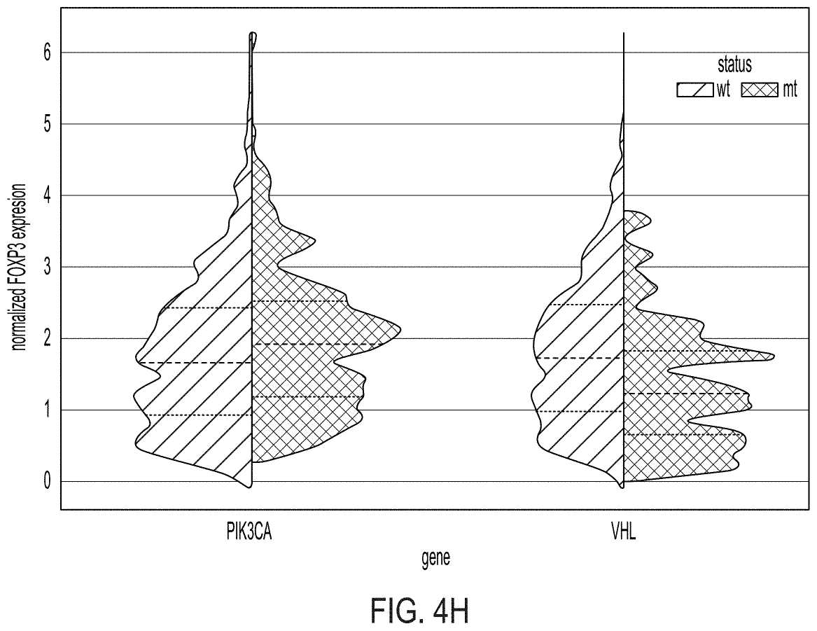

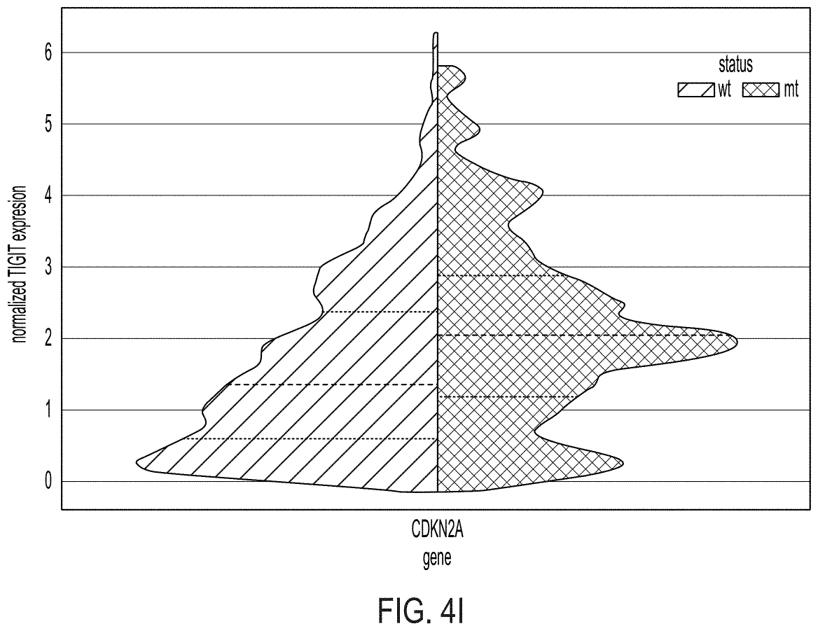

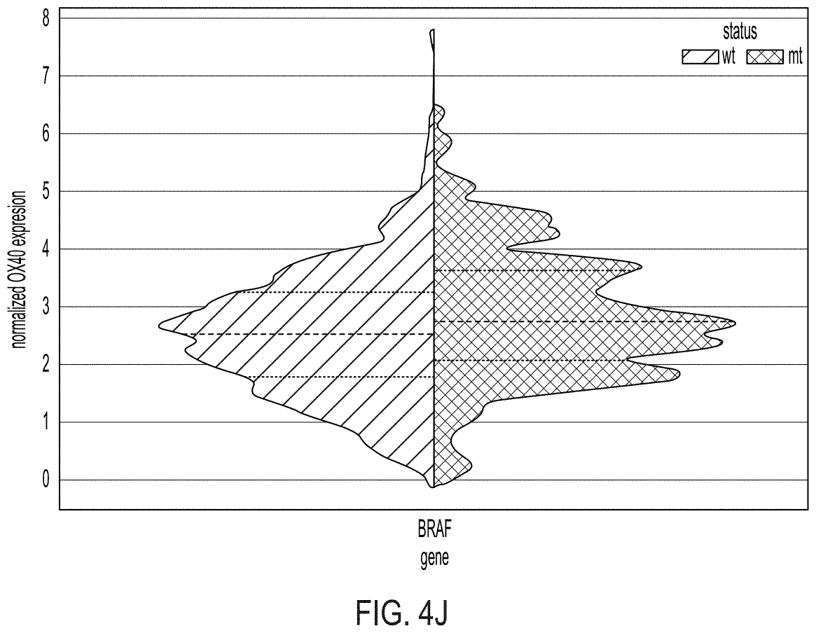

FIG. 4A-4J also illustrates mutation-associated differential checkpoint expression patterns that exceed tissue effects. Distributions for 15 tissue-independent mutation-associated checkpoint differentiators are shown. The plots for the rows in FIG. 4A-4J are same as the table in Table 2. These are the 15 combinations where the inventor has found that a combination of targeted therapy and IO therapy would be helpful for the tumor patient and would result in a synergistic effect of the two therapies.

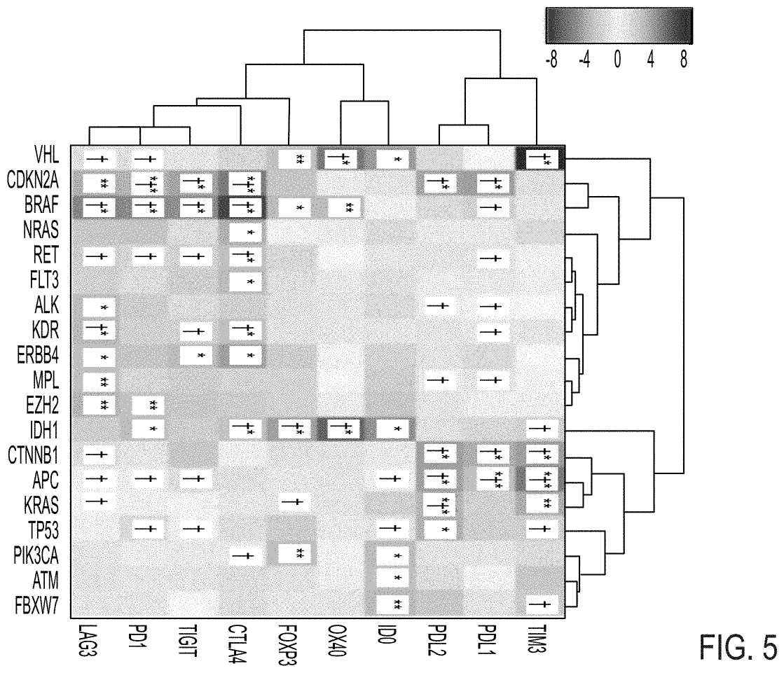

Notably, with reference to FIG. 5, using the NantHealth external database of 2739 unselected clinical cases, the associations presented in FIGS. 1-4 and in particular, FIG. 3, were validated. More specifically, 6 of the 15 combinations in FIG. 4A-4J were identified as having tissue-independent mutation-associated checkpoint differentiators in an external database.

Embodiments of the present disclosure are further described in the following examples. The examples are merely illustrative and do not in any way limit the scope of the invention as claimed.

### Example 1: Differential Expression of Immunoregulatory Molecules and Highly-Associated Cancer Genes

The use of immunotherapy in multiple cancer types is becoming mainstay along with next-generation sequencing (NGS) to identify potential actionable targets. In one embodiment, the inventors have found that some immune-regulatory molecules are often found upregulated with certain gene mutations regardless of cancer subtype.

The inventors identified 2740 TCGA patients to have at least one potentially oncogenic mutation (mt) within an established 50-gene hotspot panel, including stomach/esophageal carcinoma (N=255), skin cutaneous melanoma (N=226), stomach adenocarcinoma (N=163), breast invasive carcinoma (N=143), and lung adenocarcinoma (N=139), among others. Differential expression of 10 immunoregulatory molecules (IRM) was analyzed between mt vs. wt. To ensure observed significant associations were not confounded by tumor-type, differential IRM expression within mt-enriched tumor-types was compared to that of mt vs. wt.

19/50 gene mutations were found to be significantly associated with ≥1 IRM expression. This included elevated CTLA4 in CDKN2A mt (adj. p=1.9e-9), elevated IDO1 in FBXW7 mt (adj. p=0.007), and decreased PDL1 in APC mt (adj, p=0.02). In many, the mt effect-size was larger than that of tumor-type; e.g. head & neck carcinomas (HNSCC) are highly enriched for CDKN2A mt (OR=4.9, p=4.3e-9), yet CDKN2A mt are more associated with CTLA4 expression than HNSCC location (t=7.0 vs. 5.4). Similarly, FBXW7 mt are more associated with high IDO1 than colorectal adenocarcinoma (CRC) (t=4.3 vs. 0.9), and APC mt are more associated with low PDL1 (t=−4.1 vs. −3.2) than CRC. In total, 15 strong mt-gene/immune-regulator associations were identified.

### Example 2: Real-World Data Validation for Differential Expression of Immunoregulatory Molecules and Highly-Associated Cancer Genes

Using the NantHealth™ external database of a real-world dataset of 2739 unselected clinical cases having distinct clinicopathological characteristics, 6 of the 15 associations of Example 1 and FIGS. 1-4, were validated within the independent later-stage NantHealth cohort. With reference to FIG. 5, the 15 combinations identified in FIGS. 3 and 4 as “more significant than tissue type” are labeled with **. In the analysis of the external “real-world” dataset, the combinations that were found “more significant than tissue type” are labeled with a “+”. As such, the 6 validated out of the previously identified 15 are labeled with both a “+” and **. Additionally, the combinations in the real-world database which were found “significant” are labeled with *. Most notably, CDKN2A mt was validated as associated with increased PD1 and CTLA4 expression, while KRAS and APC mt were validated as associated with decreased PDL1/2 expression.

The presented differential checkpoint expression patterns were strongly associated with mutation status and are not primarily driven by tissue type. NGS data continues to drive agnostic approvals while immunotherapeutic efforts work to replace chemotherapy providing better efficacy with milder toxicities. This data illustrates when concomitant versus sequential therapies of genomic-driven targeted therapy and IO should be administered to tumor patients.

As used herein, the term “administering” a pharmaceutical composition or drug refers to both direct and indirect administration of the pharmaceutical composition or drug, wherein direct administration of the pharmaceutical composition or drug is typically performed by a health care professional (e.g., physician, nurse, etc.), and wherein indirect administration includes a step of providing or making available the pharmaceutical composition or drug to the health care professional for direct administration (e.g., via injection, infusion, oral delivery, topical delivery, etc.). Most preferably, the cells or exosomes are administered via subcutaneous or subdermal injection. However, in other contemplated aspects, administration may also be intravenous injection. Alternatively, or additionally, antigen presenting cells may be isolated or grown from cells of the patient, infected in vitro, and then transfused to the patient. Therefore, it should be appreciated that contemplated systems and methods can be considered a complete drug discovery system (e.g., drug discovery, treatment protocol, validation, etc.) for highly personalized cancer treatment.

As used in the description herein and throughout the claims that follow, the meaning of “a,” “an,” and “the” includes plural reference unless the context clearly dictates otherwise. Also, as used in the description herein, the meaning of “in” includes “in” and “on” unless the context clearly dictates otherwise. Unless the context dictates the contrary, all ranges set forth herein should be interpreted as being inclusive of their endpoints, and open-ended ranges should be interpreted to include commercially practical values. Similarly, all lists of values should be considered as inclusive of intermediate values unless the context indicates the contrary.

The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided with respect to certain embodiments herein is intended merely to better illuminate the full scope of the present disclosure, and does not pose a limitation on the scope of the invention otherwise claimed. No language in the specification should be construed as indicating any non-claimed element essential to the practice of the claimed invention.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

It should be apparent to those skilled in the art that many more modifications besides those already described are possible without departing from the full scope of the concepts disclosed herein. The disclosed subject matter, therefore, is not to be restricted except in the scope of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Where the specification claims refers to at least one of something selected from the group consisting of A, B, C . . . and N, the text should be interpreted as requiring only one element from the group, not A plus N, or B plus N, etc.

What is claimed is:

1. A method of identifying a tumor patient for treatment with a combination of targeted therapy and immune oncology, irrespective of the tissue type of tumor, comprising:
obtaining respective omics data for a tumor cell and a matched normal cell;
determining the expression level of a gene transcript or gene product of a tumor sample of the patient and comparing to the corresponding expression level in a matched normal sample, wherein the gene transcript or gene product is selected from the group consisting of TIM3, CTLA4, TIGIT, PDL2, PD1, IDO, OX40, PDL1, FOXP3, and/or LAG3;
determining that the patient has a mutation in a gene selected from the group consisting of APC, CDKN2A, KRAS, EZH2, FBXW7, BRAF, VHL, PIK3CA, and/or MPL; and
identifying the tumor patient for treatment with a combination of targeted therapy and immune-oncology (IO) therapy upon determination of an association of gene expression level and gene mutation.

2. The method of claim 1, wherein the difference in expression between the patient's tumor sample and the matched normal sample is at least 50%.

3. The method of claim 1, wherein the tumor is a thyroid cancer, brain cancer, liver cancer, prostate cancer, skin cancer, testicular cancer, kidney cancer, adrenal gland cancer, stomach cancer, pancreatic cancer, esophageal cancer, colon cancer, ovarian cancer, bladder cancer, uterus cancer, breast cancer, adipose tissue cancer, cervical cancer, lung cancer, muscle cancer, head and neck cancer, or bone marrow cancer.

4. The method of claim 1, wherein the tumor is stomach/esophageal carcinoma, skin cutaneous melanoma, stomach adenocarcinoma, breast invasive carcinoma, or lung adenocarcinoma.

5. The method of claim 1, wherein the matched normal sample is from the same patient.

6. The method of claim 1, wherein the matched normal sample is from a different patient.

7. The method of claim 1, wherein the expression level is determined by whole genome/exome sequencing, RNA-seq, and/or proteomic analysis of the tumor.

8. The method of claim 10, wherein the proteomic analysis is done via mass spectrometry.

9. The method of claim 1, wherein the targeted therapy comprises a therapy targeted to TIM3, CTLA4, TIGIT, LAG3, PDL2, PD1, IDO, OX40, PDL1, FOXP3, and/or LAG3.

10. The method of claim 1, wherein the IO therapy comprises treatment with T-cell therapy, and/or cancer vaccines.

11. The method of claim 1, wherein the association of gene expression level and gene mutation comprises mutation in CDKN2A gene in the tumor cell sample and PD1 overexpression.

12. The method of claim 1, wherein the association of gene expression level and gene mutation comprises mutation in CDKN2A gene in the tumor cell sample and CTLA4 overexpression.

13. The method of claim 1, wherein the association of gene expression level and gene mutation comprises mutation in CDKN2A gene in the tumor cell sample and TIGIT overexpression.

14. The method of claim 1, wherein the association of gene expression level and gene mutation comprises mutation in the APC gene in the tumor cell sample and PDL1 under-expression.

15. The method of claim 1, wherein the association of gene expression level and gene mutation comprises mutation in the APC gene in the tumor cell sample and TIM under-expression.

16. The method of claim 1, wherein the association of gene expression level and gene mutation comprises mutation in the KRAS gene in the tumor cell sample and PDL2 under-expression.

17. The method of claim 1, wherein the association of gene expression level and gene mutation comprises mutation in the KRAS gene in the tumor cell sample and TIM3 under-expression.

18. The method of claim 1, wherein the association of gene expression level and gene mutation comprises mutation in the FBXW7 gene in the tumor cell sample and IDO overexpression.

19. The method of claim 1, wherein the association of gene expression level and gene mutation comprises mutation in the EZH2 gene in the tumor cell sample and PD1 overexpression.

20. The method of claim 1, wherein the association of gene expression level and gene mutation comprises mutation in CDKN2A or EZH2 gene or MPL gene in the tumor cell sample and LAG3 over-expression.

21. The method of claim 1, wherein the association of gene expression level and gene mutation comprises mutation in PIK3CA or VHL gene in the tumor cell sample and FOXP3 over-expression.

22. The method of claim 1, wherein the association of gene expression level and gene mutation comprises mutation in BRAF gene in the tumor cell sample and OX40 under-expression.

23. The method of claim 1, wherein the association of gene expression level and gene mutation comprises mutation in the FBXW7 gene in the tumor cell sample and IDO overexpression.

24. A method of identifying correlations between specific gene mutations and expression of checkpoint inhibitors, and using the correlations to prepare a combination treatment for a tumor patient, comprising:
obtaining respective omics data for a tumor cell sample and a matched normal cell sample;
determining the expression level of an immune checkpoint inhibitor selected from the group consisting of TIM3, CTLA4, TIGIT, PDL2, PD1, IDO, OX40, PDL1, FOXP3, and/or LAG3 in the tumor sample and comparing to the expression level in a matched normal sample;
determining that the patient has a mutation in a gene selected from the group comprising APC, CDKN2A, KRAS, EZH2, FBXW7, BRAF, VHL, PIK3CA, and/or MPL; and
treating the tumor patient with a combination of targeted therapy and IO therapy upon determination of an association of gene expression level and gene mutation.

25. The method of claim 24, wherein the identification of between the specific gene mutation and expression of checkpoint inhibitors is independent of cancer type or tissue type.

26. The method of claim 24, wherein the matched normal sample is from the same patient.

27. The method of claim 24, wherein the matched normal sample is from a different patient.

28. The method of claim 24, wherein the association of gene expression level and gene mutation comprises mutation in the CDKN2A gene in the tumor cell sample and PD1 overexpression.

29. The method of claim 24, wherein the association of gene expression level and gene mutation comprises mutation in the CDKN2A gene in the tumor cell sample and CTLA4 overexpression.

30. The method of claim 24, wherein the association of gene expression level and gene mutation comprises mutation in the CDKN2A gene in the tumor cell sample and TIGIT overexpression.

31. The method of claim 24, wherein the association of gene expression level and gene mutation comprises mutation in the APC gene in the tumor cell sample and PDL1 under-expression.

32. The method of claim 24, wherein the association of gene expression level and gene mutation comprises mutation in the APC gene in the tumor cell sample and TIM3 under-expression.

33. The method of claim 24, wherein the association of gene expression level and gene mutation comprises mutation in the KRAS gene in the tumor cell sample and PDL2 under-expression.

34. The method of claim 24, wherein the association of gene expression level and gene mutation comprises mutation in the EZH2 gene in the tumor cell sample and PD1 overexpression.

35. The method of claim 24, wherein the association of gene expression level and gene mutation comprises mutation in CDKN2A or EZH2 gene or MPL gene in the tumor cell sample and LAG3 over-expression.

36. The method of claim 24, wherein the association of gene expression level and gene mutation comprises mutation in PIK3CA or VHL gene in the tumor cell sample and FOXP3 over-expression.

37. The method of claim 24, wherein the association of gene expression level and gene mutation comprises mutation in BRAF gene in the tumor cell sample and OX40 under-expression.

38. The method of claim 24, wherein the association of gene expression level and gene mutation comprises mutation in the FBXW7 gene in the tumor cell sample and IDO overexpression.

39. A method of treating a patient having a tumor, comprising:
obtaining genomics and transcriptomics data from a tumor sample of the patient and a matched normal sample;
determining the expression level, in the tumor sample and matched normal sample, of a checkpoint inhibitor selected from the group consisting of TIM3, CTLA4, TIGIT, PDL2, PD1, IDO, OX40, PDL1, FOXP3, and/or LAG3;
determining that the tumor sample has a mutation in a gene selected from the group comprising APC, CDKN2A, KRAS, EZH2, FBXW7, BRAF, VHL, PIK3CA, and/or MPL; and
treating the patient by administering a combination of (i) targeted therapy and (ii) immune-oncology (IO) therapy to the patient, upon determination of an association of gene expression level and gene mutation

40. The method of claim 39, wherein the tumor is a thyroid cancer, brain cancer, liver cancer, prostate cancer, skin cancer, testicular cancer, kidney cancer, adrenal gland cancer, stomach cancer, pancreatic cancer, esophageal cancer, colon cancer, ovarian cancer, bladder cancer, uterus cancer, breast cancer, adipose tissue cancer, cervical cancer, lung cancer, muscle cancer, head and neck cancer, or bone marrow cancer.

41. The method of claim 39, wherein the tumor is stomach/esophageal carcinoma, skin cutaneous melanoma, stomach adenocarcinoma, breast invasive carcinoma, or lung adenocarcinoma.

42. The method of claim 39, wherein the matched normal sample is from the same patient.

43. The method of claim 39, wherein the matched normal sample is from a different patient.

44. The method of claim 39, wherein the difference in expression between the patient's tumor sample and the matched normal sample is at least 50%.

45. The method of claim 39, wherein the association of gene expression level and gene mutation comprises mutation in CDKN2A in the tumor cell sample and PD1 overexpression.

46. The method of claim 39, wherein the association of gene expression level and gene mutation comprises mutation in CDKN2A in the tumor cell sample and CTLA4 overexpression.

47. The method of claim 39, wherein the association of gene expression level and gene mutation comprises mutation in CDKN2A in the tumor cell sample and TIGIT overexpression.

48. The method of claim 39, wherein the association of gene expression level and gene mutation comprises mutation in the APC gene in the tumor cell sample and PDL1 under-expression.

49. The method of claim 39, wherein the association of gene expression level and gene mutation comprises mutation in the KRAS gene in the tumor cell sample and PDL2 under-expression.

50. The method of claim 39, wherein the association of gene expression level and gene mutation comprises mutation in APC gene in the tumor cell sample and TIM3 under-expression.

51. The method of claim 39, wherein the association of gene expression level and gene mutation comprises mutation in the KRAS gene in the tumor cell sample and TIM3 under-expression.

52. The method of claim 39, wherein the association of gene expression level and gene mutation comprises mutation in EZH2 in the tumor cell sample and PD1 overexpression.

53. The method of claim 39, wherein the association of gene expression level and gene mutation comprises mutation in CDKN2A or EZH2 gene or MPL gene in the tumor cell sample and LAG3 over-expression.

54. The method of claim 39, wherein the association of gene expression level and gene mutation comprises mutation in PIK3CA or VHL gene in the tumor cell sample and FOXP3 over-expression.

55. The method of claim 39, wherein the association of gene expression level and gene mutation comprises mutation in BRAF gene in the tumor cell sample and OX40 under-expression.

56. The method of claim 39, wherein the association of gene expression level and gene mutation comprises mutation in the FBXW7 gene in the tumor cell sample and IDO overexpression.
